SEQUENCE LISTING (96 sequences)

SEQ ID NO: 1 - DNA, 20 bp, Artificial
  synthetic
gccaccgaaccagtgcctgg

SEQ ID NO: 2 - DNA, 21 bp, Artificial
  synthetic
ttactttgctactgctttgcc

SEQ ID NO: 3 - DNA, 21 bp, Artificial
  synthetic
atgaaaaagacagctatcgcg

SEQ ID NO: 4 - DNA, 35 bp, Artificial
  synthetic
ctggttcggtggcagcttgggctacggtagcgaaa

SEQ ID NO: 5 - DNA, 35 bp, Artificial
  synthetic
accgtagcccaagctgccaccgaaccagtgcctgg

SEQ ID NO: 6 - DNA, 30 bp, Artificial
  synthetic
ccggaattcttactttgctactgctttgcc

SEQ ID NO: 7 - DNA, 21 bp, Artificial
  synthetic
atgaaatacctattgcctacg

SEQ ID NO: 8 - DNA, 35 bp, Artificial
  synthetic
actggttcggtggcagccatggctggttgggcagc

SEQ ID NO: 9 - DNA, 35 bp, Artificial
  synthetic
aaccagccatggctgccaccgaaccagtgcctggc

SEQ ID NO: 10 - DNA, 30 bp, Artificial; identical to SEQ ID NO: 6
  synthetic

SEQ ID NO: 11 - DNA, 63 bp, Artificial
  synthetic
atgaaaaagacagctatcgcgattgcagtggcactggctggtttcgctaccgtagcccaagct

SEQ ID NO: 12 - DNA, 66 bp, Artificial
  synthetic
atgaaatacctattgcctacggcagccgctggattgttattactcgctgcccaaccagccatggct

SEQ ID NO: 13 - DNA, 36 bp, Artificial
  synthetic
cgccatatggagagaatgaaaaagacagctatcgcg

SEQ ID NO: 14 - DNA, 36 bp, Artificial
  synthetic
cgccatatggagagaatgaaatacctattgcctacg

SEQ ID NO: 15 - DNA, 36 bp, Artificial
  synthetic
cgccatatgagagagatgaaaaagacagctatcgcg

SEQ ID NO: 16 - DNA, 36 bp, Artificial
  synthetic
cgccatatgagagagatgaaatacctattgcctacg

SEQ ID NO: 17 - DNA, 39 bp, Artificial
  synthetic
cgccatatggagagagcgatgaaaaagacagctatcgcg

SEQ ID NO: 18 - DNA, 39 bp, Artificial
  synthetic
cgccatatggagagagcgatgaaatacctattgcctacg

SEQ ID NO: 19 - DNA, 45 bp, Artificial
  synthetic
cgccatatggaggcgagagcggcgatgaaaaagacagctatcgcg

SEQ ID NO: 20 - DNA, 45 bp, Artificial
  synthetic
cgccatatggaggcgagagcggcgatgaaatacctattgcctacg

SEQ ID NO: 21 - DNA, 48 bp, Artificial
  synthetic
cgccatatggcggcggagagagcgtgtatgaaaaagacagctatcgcg

SEQ ID NO: 22 - DNA, 48 bp, Artificial
  synthetic
cgccatatggcggcggagagagcgtgtatgaaatacctattgcctacg

SEQ ID NO: 23 - DNA, 42 bp, Artificial
  synthetic
cgccatatgagagagattgtgatgaaaaagacagctatcgcg

SEQ ID NO: 24 - DNA, 42 bp, Artificial
  synthetic
cgccatatgagagagattgtgatgaaatacctattgcctacg

SEQ ID NO: 25 - DNA, 42 bp, Artificial
  synthetic
cgccatatggagagactctgtatgaaaaagacagctatcgcg

SEQ ID NO: 26 - DNA, 42 bp, Artificial
  synthetic
cgccatatggagagactctgtatgaaatacctattgcctacg

SEQ ID NO: 27 - DNA, 51 bp, Artificial
  synthetic
cgccatatgaccagaaccgaggcgtgtgcgatgaaaaagacagctatcgcg

SEQ ID NO: 28 - DNA, 51 bp, Artificial
  synthetic
cgccatatgaccagaaccgaggcgtgtgcgatgaaatacctattgcctacg

SEQ ID NO: 29 - DNA, 54 bp, Artificial
  synthetic
cgccatatgtgtagatgtgacgcgtgtgcgctcatgaaaaagacagctatcgcg

SEQ ID NO: 30 - DNA, 54 bp, Artificial
  synthetic
cgccatatgtgtagatgtgacgcgtgtgcgctcatgaaatacctattgcctacg

SEQ ID NO: 31 - DNA, 48 bp, Artificial
  synthetic
cgccatatggagagagcgtgcgcgctcatgaaaaagacagctatcgcg

SEQ ID NO: 32 - DNA, 48 bp, Artificial
  synthetic
cgccatatggagagagcgtgcgcgctcatgaaatacctattgcctacg

SEQ ID NO: 33 - DNA, 60 bp, Artificial
  synthetic
cgccatatggtggagctcaccagagcgtgcgcgctcgcgatgaaaaagacagctatcgcg

SEQ ID NO: 34 - DNA, 60 bp, Artificial
  synthetic
cgccatatggtggagctcaccagagcgtgcgcgctcgcgatgaaatacctattgcctacg

SEQ ID NO: 35 - DNA, 63 bp, Artificial
  synthetic
cgccatatggtggcggagctcaccagagcgtgcgcgctcgcgatgaaaaagacagctatcgcg

SEQ ID NO: 36 - DNA, 63 bp, Artificial
  synthetic
cgccatatggtggcggagctcaccagagcgtgcgcgctcgcgatgaaatacctattgcctacg

SEQ ID NO: 37 - DNA, 51 bp, Artificial
  synthetic
cgccatatggagagagcgtgtgcgctcgcgatgaaaaagacagctatcgcg

SEQ ID NO: 38 - DNA, 51 bp, Artificial
  synthetic
cgccatatggagagagcgtgtgcgctcgcgatgaaatacctattgcctacg

SEQ ID NO: 39 - DNA, 57 bp, Artificial
  synthetic
cgccatatgtgtctcgagagagcgtgcgcgctcgcgatgaaaaagacagctatcgcg

SEQ ID NO: 40 - DNA, 57 bp, Artificial
  synthetic
cgccatatgtgtctcgagagagcgtgcgcgctcgcgatgaaatacctattgcctacg

SEQ ID NO: 41 - DNA, 63 bp, Artificial
  synthetic
cgccatatggtggcggagctcaccagagcgtgcgcgctcgagatgaaaaagacagctatcgcg

SEQ ID NO: 42 - DNA, 63 bp, Artificial
  synthetic
cgccatatggtggcggagctcaccagagcgtgcgcgctcgagatgaaatacctattgcctacg

SEQ ID NO: 43 - DNA, 54 bp, Artificial
  synthetic
cgccatatggaggcgagagcgtgtgtggcggtgatgaaaaagacagctatcgcg

SEQ ID NO: 44 - DNA, 54 bp, Artificial
  synthetic
cgccatatggaggcgagagcgtgtgtggcggtgatgaaatacctattgcctacg

SEQ ID NO: 45 - DNA, 51 bp, Artificial; identical to SEQ ID NO: 37
  synthetic

SEQ ID NO: 46 - DNA, 51 bp, Artificial; identical to SEQ ID NO: 38
  synthetic

SEQ ID NO: 47 - DNA, 54 bp, Artificial
  synthetic
cgccatatggacaaagcgtgctgtgtggcggtgatgaaaaagacagctatcgcg

SEQ ID NO: 48 - DNA, 54 bp, Artificial
  synthetic
cgccatatggacaaagcgtgctgtgtggcggtgatgaaatacctattgcctacg

SEQ ID NO: 49 - DNA, 60 bp, Artificial
  synthetic
cgccatatgctcgacgtgagagcgtgtgcgctcgcggcgatgaaaaagacagctatcgcg

SEQ ID NO: 50 - DNA, 60 bp, Artificial
  synthetic
cgccatatgctcgacgtgagagcgtgtgcgctcgcggcgatgaaatacctattgcctacg

SEQ ID NO: 51 - DNA, 54 bp, Artificial
  synthetic
cgccatatggagagagcgtgcgcgctcgcggcgatgaaaaagacagctatcgcg

SEQ ID NO: 52 - DNA, 54 bp, Artificial
  synthetic
cgccatatggagagagcgtgcgcgctcgcggcgatgaaatacctattgcctacg

SEQ ID NO: 53 - DNA, 69 bp, Artificial
  synthetic
cgccatatggcggcggacctcaccaaagcgtgcgcgctcgcggcggcgatgaaaaagacagctatcgcg

SEQ ID NO: 54 - DNA, 69 bp, Artificial
  synthetic
cgccatatggcggcggacctcaccaaagcgtgcgcgctcgcggcggcgatgaaatacctattgcctacg

SEQ ID NO: 55 - DNA, 57 bp, Artificial
  synthetic
cgccatatggagagagcgtgtgcgctcgcggcggcgatgaaaaagacagctatcgcg

SEQ ID NO: 56 - DNA, 57 bp, Artificial
  synthetic
cgccatatggagagagcgtgtgcgctcgcggcggcgatgaaatacctattgcctacg

SEQ ID NO: 57 - DNA, 60 bp, Artificial
  synthetic
cgccatatggagagagcgtgcgcgctcgcggcggcggcgatgaaaaagacagctatcgcg

SEQ ID NO: 58 - DNA, 60 bp, Artificial
  synthetic
cgccatatggagagagcgtgcgcgctcgcggcggcggcgatgaaatacctattgcctacg

SEQ ID NO: 59 - DNA, 75 bp, Artificial
  synthetic
cgccatatgacctgcaaatgcctcgacgcgtgcgcgctcgcggcggcggcggcgatgaaaaagacagctatcgcg

SEQ ID NO: 60 - DNA, 75 bp, Artificial
  synthetic
cgccatatgacctgcaaatgcctcgacgcgtgcgcgctcgcggcggcggcggcgatgaaatacctattgcctacg

SEQ ID NO: 61 - DNA, 66 bp, Artificial
  synthetic
cgccatatggagagactcctctgttgtaccaccaccaccaccaccatgaaaaagacagctatcgcg

SEQ ID NO: 62 - DNA, 66 bp, Artificial
  synthetic
cgccatatggagagactcctctgttgtaccaccaccaccaccaccatgaaatacctattgcctacg

SEQ ID NO: 63 - DNA, 63 bp, Artificial
  synthetic
cgccatatggagagagcgtgcgcgctcgcggcggcggcggcgatgaaaaagacagctatcgcg

SEQ ID NO: 64 - DNA, 63 bp, Artificial
  synthetic
cgccatatggagagagcgtgcgcgctcgcggcggcggcggcgatgaaatacctattgcctacg

SEQ ID NO: 65 - DNA, 75 bp, Artificial
  synthetic
cgccatatgctcaccaaatgcctcgagtgcgcgaccgcgtgttgttgttgttgtatgaaaaagacagctatcgcg

SEQ ID NO: 66 - DNA, 75 bp, Artificial
  synthetic
cgccatatgctcaccaaatgcctcgagtgcgcgaccgcgtgttgttgttgttgtatgaaatacctattgcctacg

SEQ ID NO: 67 - DNA, 63 bp, Artificial
  synthetic
cgccatatggagagaaccaccctcacctgctgctgctgctgcatgaaaaagacagctatcgcg

SEQ ID NO: 68 - DNA, 63 bp, Artificial
  synthetic
cgccatatggagagaaccaccctcacctgctgctgctgctgcatgaaatacctattgcctacg

SEQ ID NO: 69 - DNA, 72 bp, Artificial
  synthetic
cgccatatggagagagcgtgcgtggcggtggagagagcgtgcgtggcggtgatgaaaaagacagctatcgcg

SEQ ID NO: 70 - DNA, 72 bp, Artificial
  synthetic
cgccatatggagagagcgtgcgtggcggtggagagagcgtgcgtggcggtgatgaaatacctattgcctacg

SEQ ID NO: 71 - DNA, 72 bp, Artificial
  synthetic
cgccatatggagagagcgtgcgcgctcgcggtggagagagcgtgcgcgctcatgaaaaagacagctatcgcg

SEQ ID NO: 72 - DNA, 72 bp, Artificial
  synthetic
cgccatatggagagagcgtgcgcgctcgcggtggagagagcgtgcgcgctcatgaaatacctattgcctacg

SEQ ID NO: 73 - DNA, 90 bp, Artificial
  synthetic
cgccatatggagagagcgtgcgcgctcgtggagagagcgtgcgcgctcgtggagagagcgtgtgcgctcatgaaaaagacagctatcgcg

SEQ ID NO: 74 - DNA, 90 bp, Artificial
  synthetic
cgccatatggagagagcgtgcgcgctcgtggagagagcgtgcgcgctcgtggagagagcgtgtgcgctcatgaaatacctattgcctacg

SEQ ID NO: 75 - DNA, 105 bp, Artificial
  synthetic
cgccatatggagagatgcctcgcgaccctcgtggagagactctgcgtggcggtggtggagagagcgtgcgcgtgcgcgctcgcgatgaaaaagacagctatcgcg

SEQ ID NO: 76 - DNA, 105 bp, Artificial
  synthetic
cgccatatggagagatgcctcgcgaccctcgtggagagactctgcgtggcggtggtggagagagcgtgcgcgtgcgcgctcgcgatgaaatacctattgcctacg

SEQ ID NO: 77 - DNA, 30 bp, Artificial
  synthetic
cgccatatgatgaaaaagacagctatcgcg

SEQ ID NO: 78 - DNA, 30 bp, Artificial
  synthetic
cgccatatgatgaaatacctattgcctacg

SEQ ID NO: 79 - DNA, 21 bp, Artificial
  synthetic
caaacaggtggatcgtttttt

SEQ ID NO: 80 - DNA, 24 bp, Artificial
  synthetic
ttatttttttgtatagcgcaccca

SEQ ID NO: 81 - DNA, 21 bp, Artificial
  synthetic
atgaaaaagacagctatcgcg

SEQ ID NO: 82 - DNA, 36 bp, Artificial
  synthetic
aaaaaacgatccacctgtttgagcttgggctacggt

SEQ ID NO: 83 - DNA, 37 bp, Artificial
  synthetic
taccgtagcccaagctcaaacaggtggatcgtttttt

SEQ ID NO: 84 - DNA, 32 bp, Artificial
  synthetic
ccggaattttatttttttgtatagcgcaccca

SEQ ID NO: 85 - DNA, 21 bp, Artificial
  synthetic
atgaaatacctattgcctacg

SEQ ID NO: 86 - DNA, 42 bp, Artificial
  synthetic
aaaaaacgatccacctgtttgagccatggctggttgggcagc

SEQ ID NO: 87 - DNA, 37 bp, Artificial
  synthetic
ccaaccagccatggctcaaacaggtggatcgtttttt

SEQ ID NO: 88 - DNA, 32 bp, Artificial; identical to SEQ ID NO: 84
  synthetic

SEQ ID NO: 89 - DNA, 48 bp, Artificial
  synthetic
cgccatatggaacgagcatgtgttgcaatgaaaaagacagctatcgcg

SEQ ID NO: 90 - DNA, 48 bp, Artificial
  synthetic
cgccatatggaacgagcatgtgttgcaatgaaatacctattgcctacg

SEQ ID NO: 91 - DNA, 78 bp, Artificial
  synthetic
cgccatatggtggagagactatgtgtggcagtggttgaaagggcgtgtgcgctagcgatgaaaaagacagctatcgcg

SEQ ID NO: 92 - DNA, 78 bp, Artificial
  synthetic
cgccatatggtggagagactatgtgtggcagtggttgaaagggcgtgtgcgctagcgatgaaatacctattgcctacg

SEQ ID NO: 93 - DNA, 102 bp, Artificial
  synthetic
cgccatatggttgaaaggtgtctcgcgaccctcgtggagagactatgtgtggcagtggttgaaagggcgtgtgcgctagcgatgaaaaagacagctatcgcg

SEQ ID NO: 94 - DNA, 102 bp, Artificial
  synthetic
cgccatatggttgaaaggtgtctcgcgaccctcgtggagagactatgtgtggcagtggttgaaagggcgtgtgcgctagcgatgaaatacctattgcctacg

SEQ ID NO: 95 - DNA, 30 bp, Artificial; identical to SEQ ID NO: 77
  synthetic

SEQ ID NO: 96 - DNA, 30 bp, Artificial; identical to SEQ ID NO: 78
  synthetic

(19) United States
(12) Patent Application Publication
(10) Pub. No.: US 2016/0145303 A1
(43) Pub. Date: May 26, 2016

(54) Strong Secretory Signal Peptide Enhancing Small Peptide Motifs and the Use Thereof

(71) Applicant: NANJING UNIVERSITY OF TECHNOLOGY, Jiangsu (CN)

(72) Inventors: Bingfang HE, Jiangsu (CN); Wenhua CHEN, Nanjing, Jiangsu (CN); Lan MI, Nanjing, Jiangsu (CN); Shanshan WU, Nanjing, Jiangsu (CN); Yun ZHU, Nanjing, Jiangsu (CN); Shanshan CHEN, Nanjing, Jiangsu (CN)

(73) Assignee: NANJING UNIVERSITY OF TECHNOLOGY, Nanjing, Jiangsu (CN)

(21) Appl. No.: 14/786,160

(22) PCT Filed: Apr. 25, 2014
(86) PCT No.: PCT/CN2014/076249

§ 371 Date: Oct. 21, 2015

(30) Foreign Application Priority Data
May 3, 2013 (CN) 201310162455.9

(51) Int. Cl. C07K 7/06; C12P 21/02; C12N 9/24; C12N 9/46; C07K 7/08; C07K 14/00

(52) U.S. Cl. CPC C07K 7/06; C07K 7/08; C07K 14/00; C12N 9/2402; C12N 9/2454; C12P 21/02; C12Y 302/01; C12Y 302/01011; C07K 2319/02

(57) ABSTRACT

The present invention belongs to protein engineering and genetic engineering fields, relating to a strong secretory signal peptide enhancing small peptide motifs and the use thereof. The strong secretory signal peptide enhancing small peptide motifs of the present invention have the amino acid sequence of the following formula: M (αXβYγ/αYβXγ)ₙ, wherein X represents an acidic amino acid; Y represents an alkaline amino acid; α is 0 to 2 neutral amino acid(s); β represents 0 to 2 neutral amino acid(s); γ represents 1 to 10 neutral amino acid(s); n is 1 to 3. With regard to the use of the strong secretory signal peptide enhancing small peptide motifs of the present invention, it is a method for constructing a vector enhancing the secretion ability of common signal peptides to improve the secretory expression of exogenous proteins.

### CROSS-REFERENCE TO RELATED APPLICATIONS

This application claims priority from CN Application No. 201310162455.9, filed May 3, 2013 and PCT Application No. PCT/CN2014/076249, filed Apr. 25, 2014, the contents of which are incorporated herein in the entirety by reference.

### FIELD OF THE INVENTION

The present invention belongs to protein engineering and genetic engineering fields, relating to a strong secretory signal peptide enhancing small peptide motifs and the use thereof

### BACKGROUND OF THE INVENTION

The secretion system of proteins is not only the premise of protein sorting and positioning and realization of physiological function, but the important means for genetic engineering and protein engineering technology developments. People have worked out various prokaryotic and eukaryotic expression systems to produce recombinant proteins. The E. coli expression system has attracted much attention due to its clear genetic background, simple operation, rapid growth in cheap culture medium and availability for large-scale fermentation and culturing, etc.; the E. coli system has been used for preparing a variety of enzymic preparations at present, even for preparing some pharmaceutical proteins including interferon, insulin, human serum albumin, etc. and has made remarkable achievements in biotechnology field.

In principle, the recombinant proteins can be produced by the following modes in E. coli: 1. intracellular production of soluble proteins; 2. inclusion body intracellular production; 3. secreted to periplasmic space or extracellular medium. The E. coli system is applicable to the proteins which shall not be modified after production and translation. A large amount of intermediates are often accumulated to form settlement of inclusion body due to absence of cofactors required in the protein folding process of E. coli, refolding and other complex processes are also required, and the periplasmic oxidized redox environment is conductive to protein folding. If the proteins are located in the periplasmic space after crossing the intracellular membrane, even directly secreted to the extracellular medium after passing through the epicyte, the downstream refolding, separation and purification processes can be greatly simplified to reduce toxicity and metabolism burden of proteins on host bacteria and significantly improve the expression quantity.

Most existing secretory expression systems are secreted into the periplasmic space, and still need to obtain recombinant proteins by disrupting and purifying cells. A small amount of systems being secreted to the extracellular space will cause very insufficient secretion efficiency and post-processing problem of fusion proteins due to incomplete protein secretion system of E. coli itself or cognitive limitation on the secretion system.

The signal peptides are a polypeptide containing about 10-60 amino acids and generally located at N-end of secreted proteins. Using the signal peptides for the secretory expression of proteins, the general operating method is to clone the encoding gene of target proteins to the back end of the signal peptide sequence, realize fusion of target gene and signal peptide sequence at transcriptional and translational level in a host, and guide the combination of the whole polypeptide chain and intracellular molecular chaperone or secretion signal recognition particles, membrane positioning or secretion to the extracellular space by the signal peptides at N-end of the protein precursor.

At present, the E. coli system has many restrictions on extensive application of the strategy, though the extracellular secretory expression strategy has more obvious advantages in comparison with other expression modes. Thus, the prerequisite is to possess a signal peptide capable of carrying and secreting the target proteins to the extracellular space. Even if the signal peptide has the ability to mediate extracellular protein secretion, it does not indicate that a specific signal peptide for all recombinant proteins has the ability to mediate this or has the ability of the same level. Each recombinant protein has its own specific encoding gene, the transition points of the signal peptides and the recombinant proteins have difference sequences, and the structures of the recombinant proteins are different, therefore, the secretion level of the recombinant proteins depends on the optimization level of the signal peptides, transmembrane structure favorable to the target proteins and the matching level therebetween.

In the E. coli expression system, the basic secretion pathway of the secreted proteins crossing the plasma membrane is based on Sec or Tat system, e.g., outer membrane protein (OmpA) signal peptide and pectate lyase (PelB) signal peptide are secreted by Sec, trimethylamine N-oxide reductase (TorA) signal peptide is secreted by Tat, the two types of signal peptides have been successfully used in the case for the secretory expression of exogenous proteins.

With regard to incorrect or incomplete hydrolysis processing of target proteins in the yeast processing system, Patent EP 0461165 B1 has disclosed a polypeptide structure fused with hydrophobic signal peptides, hydrophilic leading peptides and heterologous proteins. The amino acid sequence between C-end of leading peptide and/or N-end of exogenous protein (near the yeast processing site) is modified to have negatively charged amino acids near the yeast processing site and facilitate full exposure of the processing site, thereby improving the shearing efficiency of protease, increasing the expression quantity of correctly processed (activated) target proteins and then secreting the target proteins to the extracellular space.

### SUMMARY OF THE INVENTION

One technical purpose of the present invention is to provide a strong secretory signal peptide enhancing small peptide motifs having function of enhancing signal peptide secretion efficiency to fuse and add the small peptide motifs to the front end of ompA signal peptide or pelB signal peptide so as to achieve the ability of the above signal peptides for the secretory expression of exogenous proteins in the E. coli system.

The other technical purpose of the present invention is to provide the use of the strong secretory signal peptide enhancing small peptide motifs having function of enhancing signal peptide secretion efficiency, i.e., a method by using the small peptide motifs for constructing a vector for enhancing the secretion ability of common signal peptides to improve the method for the secretory expression of exogenous proteins.

In the present invention, the term “secretion” means that protein or peptide molecules are transported to the outside of the bacterial cell, but it also includes the case: protein or peptide molecules finally stand in the culture medium in completely free form, and some proteins are out of the bacteria or exist in the periplasmic space of the bacteria.

The following terms used in the present invention have the following meanings, unless otherwise specified:

“Variant” of protein amino acid or polynucleotide refers to an amino acid sequence out of changes of one or more amino acid(s) or nucleotide(s) or the polynucleotide sequence for encoding amino acids or nucleotides. The said “changes” may comprise deletion, insertion or replacement of amino acids or nucleotides in the amino acid sequence or nucleotide sequence. The variant may have conservative changes, the replaced amino acids have the structure or chemical properties similar to the original amino acids, for example, isoleucine is replaced with leucine. The variant also may have non-conservative changes, for example, glycine is replaced with tryptophan.

“Deletion” refers to the deletion of one or more amino acid(s) or nucleotide(s) in the amino acid sequence or nucleotide sequence. “Insertion” or “Addition” refers to the addition of one or more amino acid(s) or nucleotide(s) due to changes in the amino acid sequence or nucleotide sequence, compared to the original molecules. “Replacement” means that one or more amino acid(s) or nucleotide(s) are replaced with different amino acids or nucleotides.

In order to achieve the technical purpose of the present invention, the technical scheme is as follows:

I. A strong secretory signal peptide enhancing small peptide motifs, characterized by comprising an amino acid sequence having the following formula: M(αXβYγ/αYβXγ)ₙ,

Wherein X represent an acidic amino acid;

Y represents an alkaline amino acid;

α is 0 to 2 neutral amino acid(s);

β represents 0 to 2 neutral amino acid(s);

γ represents 1 to 10 neutral amino acid(s);

n is 1 to 3.

Where, “/” in the formula means “or’.

Further, the acidic amino acid represented by X is preferably Glu or Asp.

Further, the basic amino acid represented by Y is preferably Arg or Lys.

Further, the neutral amino acid is preferably Ala, Cys, Leu, Val, Ile or Phe.

Further, when n=1, the small peptide motifs of the present invention have optimal enhancing secretion effect.

Further, as a most preferred embodiment of the present invention, the small peptide motifs have optimal enhancing secretion effect when α is 1 neutral amino acid, β is 0 neutral amino acid, γ is 2-5 neutral amino acids, X is Glu and Y is Arg.

Therefore, with regard to the small peptide motifs protected by the present invention and obtained by the above formula, the original small peptide motif is MERACVAV, and the derived analogues can change into:
MREACVAV
MAERACVAV;
MEARACVAV;
MDKACVAV;
MERLIVFAV; . . .

Or overlapping of small peptide motifs, e.g., MERACVA+VEARLIVFAV. See the Embodiment of the present invention for more derivation types in details.

II. The present invention also requests to protect a variant of the said strong secretory signal peptide enhancing small peptide motifs which comprise one or more amino acid residue(s) substituted for insertion or deletion of one or more amino acid residue(s) of the strong secretory signal peptide enhancing small peptide motifs and/or the amino acids with similar properties. According to the common general knowledge of a person skilled in the art, the variant still has the feature of signal peptides or function for enhancing secretion, thus, it belongs to the scope of the strong secretory signal peptide enhancing small peptide motifs the present invention requests to protect.

III. Polynucleotide of polypeptide, analogue or derivative having the said strong secretory signal peptide enhancing small peptide motifs shown in the present invention is encoded. According to the common general knowledge of a person skilled in the art, the polynucleotide of such analogue or derivative still has the feature of signal peptides or function for enhancing secretion, thus, it belongs to the scope of the strong secretory signal peptide enhancing small peptide motifs the present invention requests to protect.

IV. A recombinant vector containing exogenous polynucleotide, which is composed of the said polynucleotide and plasmid vector of III in the present invention.

V. A genetic host cell containing exogenous polynucleotide, which is transformed or transferred by the said recombinant vector of IV.

VI. For the use of the said strong secretory signal peptide enhancing small peptide motifs of the present invention, it is a method for constructing a vector enhancing the secretion ability of common signal peptides to improve the secretory expression of exogenous proteins.

Specifically, it means that the said strong secretory signal peptide enhancing small peptide motifs of the present invention are fused and added at the front end of the signal peptide, so the constructed secretion vector is transformed into the E. coli expression host for inducible expression to achieve the secretion enhancing function of the strong secretory signal peptide enhancing small peptide motifs.

In the Embodiment of the present invention, fructosidase FRU6 from Arthrobacter arilaitensis and dextranase BGL from B.subtilis are taken as target proteins, the common signal peptides are selected from ompA of outer membrane protein and pelB signal peptide of pectate lyase. After the small peptide motifs of the present invention are fused and added at the front end of ompA or pelB signal peptide, the constructed secretion vector is transformed into the E. coli expression host for inducible expression to verify the secretion enhancing function of the small peptide motifs by the testing method.

The host adopted by the present invention is E.coil BL21(DE3) which is a basic framework constructed by taking E. coli pET-22b as a vector to remove pelB signal peptide sequence in the original pET-22b. The ompA signal peptide/pelB signal peptide and fructosidase FRU6 or dextranase BGL are fused by overlapping PCR, and the upstream primers are designed to add small peptide motif gene before the signal peptide gene sequence. Secretion enhancing expression vectors pET-EompA-FRU6, pET-EpelB-FRU6, pET-EompA-BGL and pET-EpelB-BGL are constructed by enzyme cutting and connection at the enzyme cutting site.

The recombinant plasmid vectors pET-EompA-FRU6 and pET-EpelB-FRU6 (or pET-EompA-BGL and pET-EpelB-BGL) containing fused secretion enhancing signal peptide and fructosidase FRU6 (or dextranase BGL) are respectively named as BL21/pET-EompA-FRU6 and BL21/pET-EpelB-FRU6 (or BL21/pET-EompA-BGL and BL21/pET-EpelB-BGL) after being transformed into the host E. coli BL21. The inducer is IPTG when LB culture medium is subject to the inducible expression.

Results obtained after the secretory expression of the above two target proteins are acceptable in the testing on enzyme activities of intracellular and extracellular culture media and analysis on SDS-PAGE.

The secretion examples adopted by the present invention include fructosidase FRU6 and β-1,3-1,4 dextranase BGL, Fructosidase FRU6 (molecular weight is about 55 kDa from Arthrobacter arilaitensis NJEM01, strain Collection No. CCTCC M 2012155). The classification No. of the fructosidase system is EC 3.2.1.80 which is an extracellular enzyme, so 2,1-β-glucosidic bond or 2,6-β-glucosidic bond at the non-reducing end of fructosan molecule composed of β-D-fructose can be specifically catalyzed and hydrolyzed, in addition, synanthrin, sucrose, raffinose, etc. also can be hydrolyzed. Such extracellular enzymes have high utilization value in biological, medical and food fields. Dextranase BGL (molecular weight is about 28 kDa, EC 3.2.1.73, β-dextranase is from B. subtilis) is an incision hydrolase which can efficiently and metastatically hydrolyze β-1,4 glucosidic bond close to β-1,3 glucosidic bond in β-glucan, thereby reducing adverse impact of β-glucan in cereals on industrial production. It also has very important application value in beer brewing industry, feed industry and other fields.

In addition, the beneficial effects of the present invention are as follows: the small peptide motifs capable of enhancing the secretion function are connected with N-end of the signal peptide and have obvious secretion enhancing effect on target proteins. The test on the secretory expression example of fructosidase FRU6 shows that the secretion efficiency is increased by 5.1 times to the utmost extent after addition of the small peptide motifs before ompA signal peptide and the secretion efficiency is increased by 5.4 times to the utmost extent after addition of the small peptide motifs before pelB signal peptide. The test on the secretory expression example of dextranase BGL shows that the secretion efficiency of the small peptide motifs is increased by 2.3 times compared with that of ompA signal peptide and 2.5 times compared with that of PelB signal peptide. With regard to the polypeptide structure disclosed by EP 0461165 B1, it is to fully expose the yeast processing site by modifying the amino acid sequence between C-end of leading peptide and/or N-end of exogenous protein, thereby promoting the correct processing of target proteins and improving the active protein expression quantity. In addition, the secretory expression vector constructed in the present invention can be used to produce a variety of recombinant proteins.

### BRIEF DESCRIPTION OF THE DRAWINGS

FIG. 1 is a map of the recombinant vector pET-EompA-FRU6;

Wherein ompA SP is the signal peptide encoding sequence of outer membrane protein A; FRU6 is the encoding sequence of fructosidase FRU6; Amp sequence is the resistant gene encoding sequence of ampicillin and T7 is the promoter.

FIG. 2 is the relationship between the secretion enhancing efficiency of small peptide motifs and the fermentation time.

FIG. 3 is the activity comparison between BL21/pET-EompA-FRU6 and BL21/pET-EpelB-FRU6 in the culture medium and negative control fermenting supernatant enzyme.

FIG. 4 is an electrophoretogram of SDS-PAGE of BL21/pET-EompA-FRU6 fermentation liquor in the culture medium;

Wherein M is the marker of protein; 1 and 2 are the negative controls for BL21 strain fermenting supernatant and thallus fragmentized supernatant without signal peptide sequence before fructosidase FRU6; 3 and 4 are BL21/pET-ompA-FRU6 strain fermenting supernatant and thallus fragmentized supernatant. 5 and 6 are BL21/pET-EompA-FRU6 strain fermenting supernatant and thallus fragmentized supernatant. (In pET-EompA-FRU6 plasmid, the small peptide motif sequence added before ompA signal peptide is MERACALA). The arrow direction is the molecular weight position for the mature peptide of fructosidase FRU6.

FIG. 5 is an electropherogram of SDS-PAGE of BL21/pET-EpelB-FRU6 fermentation liquor in LB culture medium.

Wherein M is the marker of protein; 1 and 2 are the negative controls for BL21 strain fermenting supernatant and thallus fragmentized supernatant without signal peptide sequence before fructosidase FRU6; 3 and 4 are BL21/pET-pelB-FRU6 strain fermenting supernatant and thallus fragmentized supernatant. 5 and 6 are BL21/pET-EpelB-FRU6 strain fermenting supernatant and thallus fragmentized supernatant. (In pET-EpelB-FRU6 plasmid, the small peptide motif sequence added before pelB signal peptide is MERACALA). The arrow direction is the molecular weight position for the mature peptide of fructosidase FRU6.

FIG. 6 is an electrophoretogram of SDS-PAGE of BL21/pET-EompA-BGL fermentation liquor in the culture medium;

Wherein M is the marker of protein; 1 and 2 are the negative controls for BL21 strain fermenting supernatant and thallus fragmentized supernatant without signal peptide sequence before dextranase BGL; 3 and 4 are BL21/pET-ompA-BGL strain fermenting supernatant and thallus fragmentized supernatant. 5 and 6 are BL21/pET-EompA-BGL strain fermenting supernatant and thallus fragmentized supernatant. (In pET-EompA-BGL plasmid, the small peptide motif sequence added before ompA signal peptide is MERACALA). The arrow direction is the molecular weight position for the mature peptide of dextranase BGL.

FIG. 7 is an electropherogram of SDS-PAGE of BL21/pET-EpelB-BGL fermentation liquor in LB culture medium.

Wherein M is the marker of protein; 1 and 2 are the negative controls for BL21 strain fermenting supernatant and thallus fragmentized supernatant without signal peptide sequence before dextranase BGL; 3 and 4 are BL21/pET-pelB-BGL strain fermenting supernatant and thallus fragmentized supernatant; 5 and 6 are BL21/pET-EpelB-BGL strain fermenting supernatant and thallus fragmentized supernatant. (In pET-EpelB-BGL plasmid, the small peptide motif sequence added before pelB signal peptide is MERACALA). The arrow direction is the molecular weight position for the mature peptide of dextranase BGL.

### EMBODIMENTS

Unless otherwise specified, the methods of the following embodiments are conventional methods, and the involved plasmid, reagent and other materials can be commercially obtained.

### Embodiment 1

### Use of Small Peptide Enhancing Motifs for Secretory Expression of Fructosidase

First, the said fructosidase Fru6 gene in the Embodiment is from Arthrobacter arilaitensis NJEM01 strain which is previously applied by the inventor in China, the patent application No. is CN102732456A and the Collection No. of the strain is CCTCC NO: M 2012155.

All primers involved in the Embodiment are synthesized by Invitrogen Company. See Table 1. The following primers are uniformly expressed as “P” plus No., for example, for the No. 45 primer in Table 2, the code is P45 and the No. in the sequence table is SEQ ID NO: 45.

TABLE 1
Primers required for Embodiment 1 and nucleotide
sequence of synthetic sequence

| SEQ ID NO | Nucleotide sequence of primer (5′-3′) |
|---|---|
| 1 | GCCACCGAACCAGTGCCTGG |
| 2 | TTACTTTGCTACTGCTTTGCC |
| 3 | ATGAAAAAGACAGCTATCGCG |
| 4 | CTGGTTCGGTGGCAGCTTGGGCTACGGTAGCGAAA |
| 5 | ACCGTAGCCCAAGCTGCCACCGAACCAGTGCCTGG |
| 6 | CCGGAATTC TTACTTTGCTACTGCTTTGCC |
| 7 | ATGAAATACCTATTGCCTACG |
| 8 | ACTGGTTCGGTGGCAGCCATGGCTGGTTGGGCAGC |
| 9 | AACCAGCCATGGCTGCCACCGAACCAGTGCCTGGC |
| 10 | CCGGAATTC TTACTTTGCTACTGCTTTGCC |

Note:
the underlined part in the table is shown as the restriction enzyme cutting site.

(1) Obtaining of fructosidase FRU6 gene: taking the genome of Arthrobacter arilaitensis NJEM01 strain as a template, primers P1 and P2 are used for PCR reaction so as to amplify the gene segment of fructosidase FRU6 (excluding the signal peptide sequence of the enzyme itself), the segment is cloned to the clone vector pMD18-T to obtain pMD-T-FRU6 and transformed into the clone host DH5α, therefore, the testing on DNA sequence can validate the correctness.

(2) Fusion of signal peptide and fructosidase gene: ompA and pelB signal peptide gene sequences (i.e., primer P11 and primer P12) are artificially synthesized to extract the plasmid pMD-T-FRU6 from Step (1). PCR primers P3-P6 or P7-P10 are overlapped after being designed, fructosidase FRU6 and signal peptide ompA or pelB are fused respectively. P6 and P10 all have EcoRI enzyme cutting sites.

(3) Design of secretion enhancing vector:

The amino acid sequence of small peptide motif is taken as MERACALA. See Table 2 and Table 3 for more small peptide motifs.

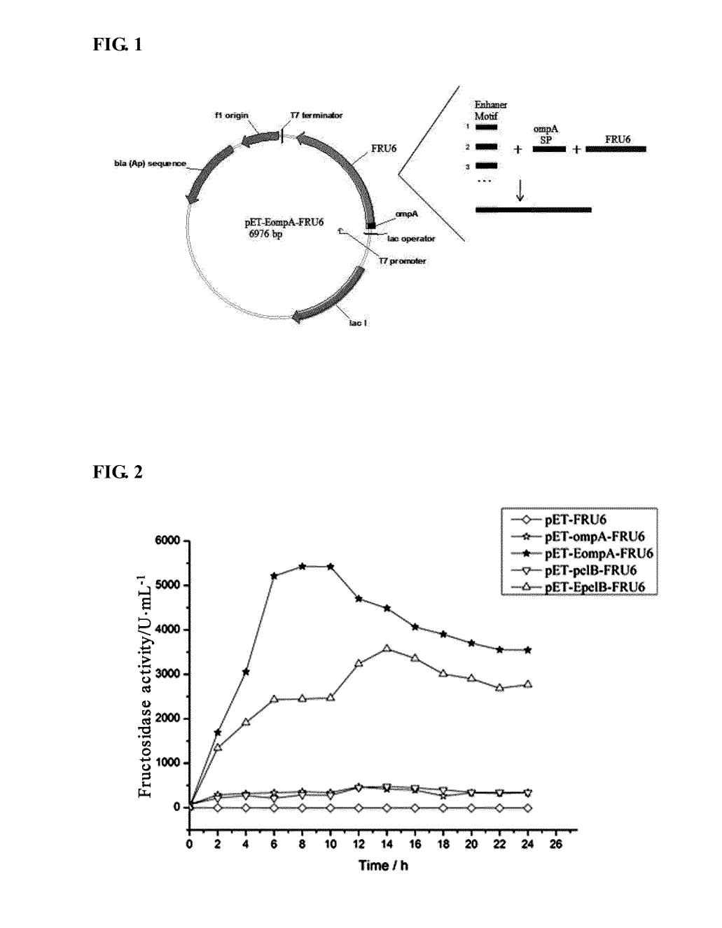

The fusion gene in Step (2) is taken as a template, and upstream primers P45 and P46 have NdeI enzyme cutting site after being designed. Upon common PCR reaction, primers P45 and P6 are combined to add the small peptide motif gene before ompA signal peptide gene; primers P46 and P10 are combined to add the small peptide motif gene (e.g., the amino sequence of small peptide motifs is MERACALA) before pelB signal peptide gene. NdeI and EcoRI double-enzyme digestion is used for obtaining the target segments at the cohesive end. Vector pET-22b (purchased from Invitrogen Company) is subject to enzyme cutting degradation by restriction enzymes NdeI and EcoRI to recover and remove large plasmid segments of pelB signal peptide sequence. Then the recovered large plasmid segments and the enzyme cutting product of secretion enhancing signal peptide and fructosidase fusion gene are connected by T4 ligase to obtain pET-EompA-FRU6 (see FIG. 1 for the plasmid map) and pET-EpelB-FRU6 (neglected when the plasmid map is similar to the former map) and transformed into the clone host E. coli DH5α, so the testing on DNA sequence can validate the correctness.

TABLE 2
Amino acid sequences, primers and enzyme
activities of secretion enhancing small
peptide motifs involved in corresponding
fructosidase

| | | | X | | Y | | | | |
|---|---|---|---|---|---|---|---|---|---|
| | | | (or | | (or | | | Signal | Enzyme |
| n | M | α | Y) | β | X) | γ | Primer | peptide | activity |
| 1 | M | | E | | R | | 13 | ompA | 1279 |
| | | | | | | | 14 | pelB | 1521 |
| 1 | M | | R | | E | | 15 | ompA | 801 |
| | | | | | | | 16 | pelB | 917 |
| 1 | M | | E | | R | A | 17 | ompA | 1831 |
| | | | | | | | 18 | pelB | 1734 |
| 1 | M | | E | A | R | AA | 19 | ompA | 1678 |
| | | | | | | | 20 | pelB | 1530 |
| 1 | M | AA | E | | R | AC | 21 | ompA | 2169 |
| | | | | | | | 22 | pelB | 1781 |
| 1 | M | | R | | E | IV | 23 | ompA | 907 |
| | | | | | | | 24 | pelB | 862 |
| 1 | M | | E | | R | LC | 25 | ompA | 2380 |
| | | | | | | | 26 | pelB | 1972 |
| 1 | M | T | R | T | E | ACA | 27 | ompA | 2024 |
| | | | | | | | 28 | pelB | 1976 |
| 1 | M | C | R | C | D | ACAL | 29 | ompA | 2175 |
| | | | | | | | 30 | pelB | 2005 |
| 1 | M | | E | | R | ACAL | 31 | ompA | 2169 |
| | | | | | | | 32 | pelB | 1781 |
| 1 | M | V | E | LT | R | ACALA | 33 | ompA | 2513 |
| | | | | | | | 34 | pelB | 2100 |
| 1 | M | | E | | R | ACALA | 35 | ompA | 1877 |
| | | | | | | | 36 | pelB | 1762 |
| 1 | M | CL | E | | R | ACALA | 37 | ompA | 2395 |
| | | | | | | | 38 | pelB | 2230 |
| 1 | M | V | E | T | R | ACALA | 39 | ompA | 2460 |
| | | | | | | | 40 | pelB | 2195 |
| 1 | M | VA | E | LT | R | ACALA | 41 | ompA | 2380 |
| | | | | | | | 42 | pelB | 2120 |
| 1 | M | | E | A | R | ACVAV | 43 | ompA | 2390 |
| | | | | | | | 44 | pelB | 2275 |
| 1 | M | | E | | R | ACALA | 45 | ompA | 2016 |
| | | | | | | | 46 | pelB | 1979 |
| | M | | D | | K | ACVAV | 47 | ompA | 1193 |
| | | | | | | | 48 | pelB | 1272 |
| 1 | M | L | D | V | R | ACALAA | 49 | ompA | 1754 |
| | | | | | | | 50 | pelB | 1644 |
| 1 | M | | E | | R | ACALAA | 51 | ompA | 1980 |
| | | | | | | | 52 | pelB | 1642 |
| 1 | M | AA | D | LT | K | ACALAAA | 53 | ompA | 1766 |
| | | | | | | | 54 | pelB | 1790 |
| 1 | M | | E | | R | ACALAAA | 55 | ompA | 1987 |
| | | | | | | | 56 | pelB | 1755 |
| 1 | M | | E | | R | ACALAAAA | 57 | ompA | 2485 |
| | | | | | | | 58 | 1pelB | 1643 |
| 1 | M | TC | K | CL | D | ACALAAAAA | 59 | ompA | 1906 |
| | | | | | | | 60 | pelB | 1560 |
| 1 | M | | E | | R | LLCCTTTTT | 61 | ompA | 1986 |
| | | | | | | | 62 | pelB | 1756 |
| 1 | M | | E | | R | ACALAAAAA | 63 | o1mpA | 1762 |
| | | | | | | | 64 | pelB | 1882 |
| 1 | M | LT | K | CL | E | CATACCCCCC | 65 | ompA | 1883 |
| | | | | | | | 66 | pelB | 1986 |
| 1 | M | | E | | R | TTLTCCCCCC | 67 | ompA | 1880 |
| | | | | | | | 68 | pelB | 1670 |

| | | | | |
|---|---|---|---|---|
| 2 | MERACVAV + MERACVAV | 69 | ompA | 1787 |
| | | 70 | pelB | 1754 |
| 2 | MERACALA + VERACAL | 71 | ompA | 2460 |
| | | 72 | pelB | 1845 |
| 3 | MERACAL + VERACAL + | 73 | ompA | 1680 |
| | VERACAL | 74 | pelB | 1855 |
| 3 | MERCLATL + VERLCVAV + | 75 | ompA | 2260 |
| | VERACALA | 76 | pelB | 2042 |
| 0 | Blank | 77 | ompA | 420 |
| | | 78 | pelB | 495 |

Note:
the primer No. in the table corresponds to P or
SEQ ID NO. OmpA and PelB signal peptide sequences
are as follows:
OmpA: ATGAAAAAGACAGCTATCGCGATTGCAGTGGCACTGGCTGGTTT
CGCTACCGTAGCCCAAGCT (SEQ ID NO: 11);
PelB: ATGAAATACCTATTGCCTACGGCAGCCGCTGGATTGTTATTACT
CGCTGCCCAACCAGCCATGGT (SEQ ID NO: 12),

TABLE 3
Upstream primers correspondingly designed for the
said small peptide motifs of Table 2

| Primer | Sequence (5′→3′) |
|---|---|
| 13 | CGCCATATGGAGAGA ATGAAAAAGACAGCTATCGCG |
| 14 | CGCCATATGGAGAGAATGAAATACCTATTGCCTACG |
| 15 | CGCCATATGAGAGAG ATGAAAAAGACAGCTATCGCG |
| 16 | CGCCATATGAGAGAGATGAAATACCTATTGCCTACG |
| 17 | CGCCATATGGAGAGAGCG ATGAAAAAGACAGCTATCGCG |
| 18 | CGCCATATGGAGAGAGCGATGAAATACCTATTGCCTACG |
| 19 | CGCCATATGGAGGCGAGAGCGGCG ATGAAAAAGACAGCTAT |
| | CGCG |
| 20 | CGCCATATGGAGGCGAGAGCGGCGATGAAATACCTATTGCCT |
| | ACG |
| 21 | CGCCATATGGCGGCGGAGAGAGCGTGT ATGAAAAAGACAGC |
| | TATCGCG |
| 22 | CGCCATATGGCGGCGGAGAGAGCGTGTATGAAATACCTATTG |
| | CCTACG |
| 23 | CGCCATATGAGAGAGATTGTG ATGAAAAAGACAGCTATCGC |
| | G |
| 24 | CGCCATATGAGAGAGATTGTGATGAAATACCTATTGCCTACG |
| 25 | CGCCATATGGAGAGACTCTGT ATGAAAAAGACAGCTATCGC |
| | G |
| 26 | CGCCATATGGAGAGACTCTGTATGAAATACCTATTGCCTACG |
| 27 | CGCCATATGACCAGAACCGAGGCGTGTGCGATGAAAAAGACA |
| | GCTATCGCG |
| 28 | CGCCATATGACCAGAACCGAGGCGTGTGCGATGAAATACCTA |
| | TTGCCTACG |
| 29 | CGCCATATGTGTAGATGTGACGCGTGTGCGCTC ATGAAAAA |
| | GACAGCTATCGCG |
| 30 | CGCCATATGTGTAGATGTGACGCGTGTGCGCTCATGAAATAC |
| | CTATTGCCTACG |
| 31 | CGCCATATGGAGAGAGCGTGCGCGCTC ATGAAAAAGACAGC |
| | TATCGCG |
| 32 | CGCCATATGGAGAGAGCGTGCGCGCTCATGAAATACCTATTG |
| | CCTACG |
| 33 | CGCCATATGGTGGAGCTCACCAGAGCGTGCGCGCTCGCG AT |
| | GAAAAAGACAGCTATCGCG |
| 34 | CGCCATATGGTGGAGCTCACCAGAGCGTGCGCGCTCGCGATG |
| | AAATACCTATTGCCTACG |
| 35 | CGCCATATGGTGGCGGAGCTCACCAGAGCGTGCGCGCTCGCG |
| | ATGAAAAAGACAGCTATCGCG |
| 36 | CGCCATATGGTGGCGGAGCTCACCAGAGCGTGCGCGCTCGCG |
| | ATGAAATACCTATTGCCTACG |
| 37 | CGCCATATGGAGAGAGCGTGTGCGCTCGCG ATGAAAAAGAC |
| | AGCTATCGCG |
| 38 | CGCCATATGGAGAGAGCGTGTGCGCTCGCGATGAAATACCTA |
| | TTGCCTACG |
| 39 | CGCCATATGTGTCTCGAGAGAGCGTGCGCGCTCGCG ATGAA |
| | AAAGACAGCTATCGCG |
| 40 | CGCCATATGTGTCTCGAGAGAGCGTGCGCGCTCGCGATGAAA |
| | TACCTATTGCCTACG |
| 41 | CGCCATATGGTGGCGGAGCTCACCAGAGCGTGCGCGCTCGAG |
| | ATGAAAAAGACAGCTATCGCG |
| 42 | CGCCATATGGTGGCGGAGCTCACCAGAGCGTGCGCGCTCGAG |
| | ATGAAATACCTATTGCCTACG |
| 43 | CGCCATATGGAGGCGAGAGCGTGTGTGGCGGTG ATGAAAAA |
| | GACAGCTATCGCG |
| 44 | CGCCATATGGAGGCGAGAGCGTGTGTGGCGGTGATGAAATAC |
| | CTATTGCCTACG |
| 45 | CGCCATATGGAGAGAGCGTGTGCGCTCGCG ATGAAAAAGAC |
| | AGCTATCGCG |
| 46 | CGCCATATGGAGAGAGCGTGTGCGCTCGCGATGAAATACCTA |
| | TTGCCTACG |
| 47 | CGCCATATGGACAAAGCGTGCTGTGTGGCGGTG ATGAAAAA |
| | GACAGCTATCGCG |
| 48 | CGCCATATGGACAAAGCGTGCTGTGTGGCGGTGATGAAATAC |
| | CTATTGCCTACG |
| 49 | CGCCATATGCTCGACGTGAGAGCGTGTGCGCTCGCGGCG AT |
| | GAAAAAGACAGCTATCGCG |
| 50 | CGCCATATGCTCGACGTGAGAGCGTGTGCGCTCGCGGCGATG |
| | AAATACCTATTGCCTACG |
| 51 | CGCCATATGGAGAGAGCGTGCGCGCTCGCGGCG ATGAAAAA |
| | GACAGCTATCGCG |
| 52 | CGCCATATGGAGAGAGCGTGCGCGCTCGCGGCGATGAAATAC |
| | CTATTGCCTACG |
| 53 | CGCCATATGGCGGCGGACCTCACCAAAGCGTGCGCGCTCGCG |
| | GCGGCGATGAAAAAGACAGCT |
| | ATCGCG |
| 54 | CGCCATATGGCGGCGGACCTCACCAAAGCGTGCGCGCTCGCG |
| | GCGGCGATGAAATACCTATTGC |
| | CTACG |
| 55 | CGCCATATGGAGAGAGCGTGTGCGCTCGCGGCGGCG ATGAA |
| | AAAGACAGCTATCGCG |
| 56 | CGCCATATGGAGAGAGCGTGTGCGCTCGCGGCGGCGATGAAA |
| | TACCTATTGCCTACG |
| 57 | CGCCATATGGAGAGAGCGTGCGCGCTCGCGGCGGCGGCG AT |
| | GAAAAAGACAGCTATCGCG |
| 58 | CGCCATATGGAGAGAGCGTGCGCGCTCGCGGCGGCGGCGATG |
| | AAATACCTATTGCCTACG |
| 59 | CGCCATATGACCTGCAAATGCCTCGACGCGTGCGCGCTCGCG |
| | GCGGCGGCGGCGATGAAAAAGACAGCTATCGCG |
| 60 | CGCCATATGACCTGCAAATGCCTCGACGCGTGCGCGCTCGCG |
| | GCGGCGGCGGCGATGAAATACCTATTGCCTACG |
| 61 | CGCCATATGGAGAGACTCCTCTGTTGTACCACCACCACCACC |
| | ACCATGAAAAAGACAGCTATCGCG |
| 62 | CGCCATATGGAGAGACTCCTCTGTTGTACCACCACCACCACC |
| | ACCATGAAATACCTATTGCCTACG |
| 63 | CGCCATATGGAGAGAGCGTGCGCGCTCGCGGCGGCGGCGGCG |
| | ATGAAAAAGACAGCTATCGCG |
| 64 | CGCCATATGGAGAGAGCGTGCGCGCTCGCGGCGGCGGCGGCG |
| | ATGAAATACCTATTGCCTACG |
| 65 | CGCCATATGCTCACCAAATGCCTCGAGTGCGCGACCGCGTGT |
| | TGTTGTTGTTGTATGAAAAAGACAGCTATCGCG |
| 66 | CGCCATATGCTCACCAAATGCCTCGAGTGCGCGACCGCGTGT |
| | TGTTGTTGTTGTATGAAATACCTATTGCCTACG |
| 67 | CGCCATATGGAGAGAACCACCCTCACCTGCTGCTGCTGCTGC |
| | ATGAAAAAGACAGCTATCGCG |
| 68 | CGCCATATGGAGAGAACCACCCTCACCTGCTGCTGCTGCTGC |
| | ATGAAATACCTATTGCCTACG |
| 69 | CGCCATATGGAGAGAGCGTGCGTGGCGGTGGAGAGAGCGTGC |
| | GTGGCGGTGATGAAAAAGACAGCTATCGCG |
| 70 | CGCCATATGGAGAGAGCGTGCGTGGCGGTGGAGAGAGCGTGC |
| | GTGGCGGTGATGAAATACCTATTGCCTACG |
| 71 | CGCCATATGGAGAGAGCGTGCGCGCTCGCGGTGGAGAGAGCG |
| | TGCGCGCTCATGAAAAAGACAGCTATCGCG |
| 72 | CGCCATATGGAGAGAGCGTGCGCGCTCGCGGTGGAGAGAGCG |
| | TGCGCGCTCATGAAATACCTATTGCCTACG |
| 73 | CGCCATATGGAGAGAGCGTGCGCGCTCGTGGAGAGAGCGTGC |
| | GCGCTCGTGGAGAGAGCGTGTGCGCTC ATGAAAAAGACAGC |
| | TATCGCG |
| 74 | CGCCATATGGAGAGAGCGTGCGCGCTCGTGGAGAGAGCGTGC |
| | GCGCTCGTGGAGAGAGCGTGTGCGCTCATGAAATACCTATTG |
| | CCTACG |
| 75 | CGCCATATGGAGAGATGCCTCGCGACCCTCGTGGAGAGACTC |
| | TGCGTGGCGGTGGTGGAGAGAGCGTGCGCGTGCGCGCTCGCG |
| | ATGAAAAAGACAGCTATCGCG |
| 76 | CGCCATATGGAGAGATGCCTCGCGACCCTCGTGGAGAGACTC |
| | TGCGTGGCGGTGGTGGAGAGAGCGTGCGCGTGCGCGCTCGCG |
| | ATGAAATACCTATTGCCTACG |
| 77 | CGCCATATGATGAAAAAGACAGCTATCGCG |
| 78 | CGCCATATGATGAAATACCTATTGCCTACG |

Note:
the primer No. in the table corresponds to P or SEQ ID NO.

(4) Construction of secretion enhancing expression strain: pET-EompA-FRU6 and pET-EpelB-FRU6 vectors are transformed into the host BL21. See Molecular Cloning Manual for the operating method. The E. coli BL21 recombinant strains containing pET-EompA-FRU6 and pET-EpelB-FRU6 vectors are screened on LB plate containing 100 μg/mL ampicillin, and named as BL21/pET-EompA-FRU6 and BL21/pET-EpelB-FRU6.

(5) Secretory expression of fructosidase FRU6: the recombinant strain is inoculated to LB liquid culture medium containing 100 μg/mL ampicillin at 37° C., and inoculated to the fresh LB culture medium (containing 100 μg/mL ampicillin) at 37° C. as per 2% inoculation amount after being cultured overnight at 200 rpm. Inducer IPTG (final concentration is 1 mmol/L) is added when OD₆₀₀ is 0.6-0.9 after being cultured at 200 rpm, and samples are collected every two hours.

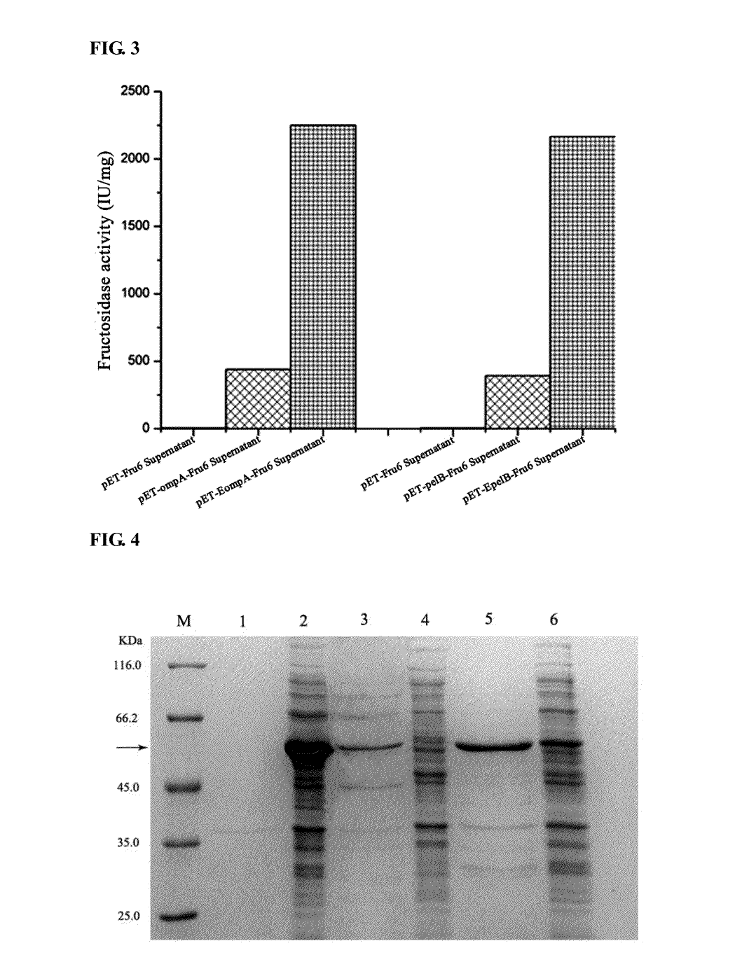

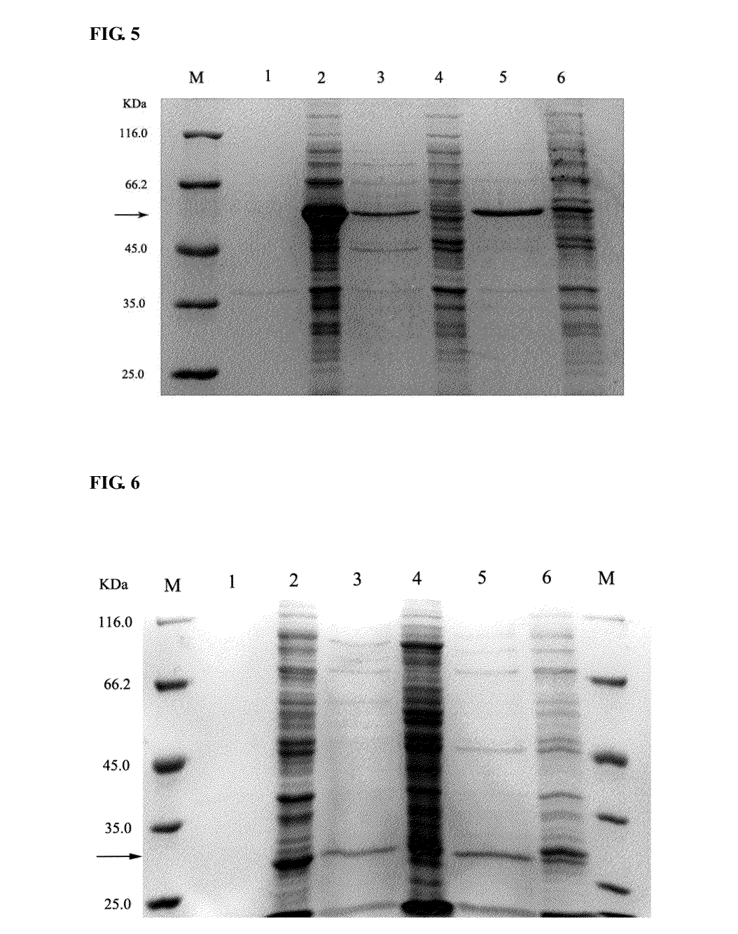

(6) Testing on enzyme activity: supernatant is taken from centrifuging cells and acellular fragmentized liquid is taken from disrupted cells after the recombinant strains are fermented in a shake flask. BL21/pET-EompA-FRU6 and BL21/pET-EpelB-FRU6 fermenting supernatants and intracellular enzyme activities are tested, and the recombinant strain without enhancing small peptide motifs is taken as a negative control. See FIG. 2 (IPTG induction 0-24 hour(s), comparison between secretion efficiencies of supernatant and fructosidase after fermentation of BL21 hosts with small peptide motifs and without small peptide motifs and signal peptides) and FIG. 3 (fermentation and sampling by the shake flask for 6 hours in LB culture medium as per 2% inoculation amount) for results. Under unit thallus mass (mg) condition, BL21/pET-EompA-FRU6 or BL21/pET-EpelB-FRU6 is compared with the negative control fermenting supernatant enzyme in activity (see Table 2 for the enhancing effect of more small peptide motifs, each small peptide motif is fermented 6 hours and sampled for testing of enzyme activity, samples taken in other times are not listed one by one).

Testing method of enzyme activity is as follows: \textcircled{1} preparation of substrate: adequately dissolve 0.1 g puerarin and 2.8 g sucrose into 100 mL 0.05 mol/L and pH6 phosphate buffer; \textcircled{2} reaction system: add 50 μL phosphate buffer (0.05 mol/L, pH 6) to 950 substrate, stand at 35° C. for reaction 10 min, and then immediately take 100 μL and add to 900 μL methanol for termination of reaction, and take it as blank control, add another 50 μL enzyme liquid to 950 μL substrate, stand at 35° C. for reaction 10 min, finally take out 100 μL and add to 900 μL methanol for termination of reaction, take it as a sample and test the sample with HPLC; \textcircled{3} definition of enzyme activity unit: take the quantity of enzyme required for consumption of 1 μg puerarin at 35° C. every minute as an active unit (U).

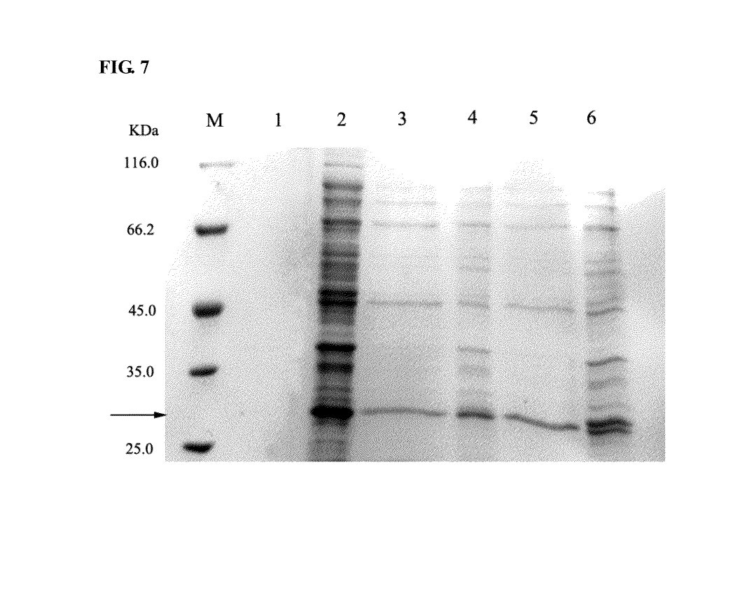

(7) SDS-PAGE polyacrylamide gel electrophoresis: see Molecular Cloning Manual for the operating method, and see FIG. 4 and FIG. 5 for results.

According to the above enzyme activity testing method, it can be seen from Table 2 and Table 3 that the enzyme activity is obviously enhanced after the said small peptide of the present invention in Table 2 is fused at the front end of the signal peptide of ompA or pelB adopted for the secretory expression of fructosidase FRU6, it is shown that the said small peptide enhancing motifs of the present invention have extremely strong secretory expression enhancing ability.

### Embodiment 2

### Use of Small Peptide Enhancing Motifs for Secretory Expression of Dextranase

(1) Construction of the expression vector of small peptide enhancing motifs is added before different signal peptides at N-end of dextranase BGL

Same as Embodiment 1, fructosidase of Embodiment 1 is replaced with dextranase gene in the Embodiment.

Wherein the sequence of dextranase BGL is from Bacillus subtilis subsp. subtilis 6051-HGW, the sequence No. of GenBank is CP003329.1 and the scope is from 4011849 to 4012490.

The gene segment of dextranase BGL amplified by PCR reaction of primers P79 and P80 is prepared to be T vector pMD-T-BGL. Primers P81-P84 or P85-P88 for PCR are overlapped to fuse dextranase BGL and signal peptide ompA or pelB respectively. R84 and R88 all have EcoRI enzyme cutting sites. The upstream primers P89-P96 are combined with the downstream primer P84 or P88, and different enhancing motifs can be designed and fused before ompA or pelB signal peptide.

All primers involved in the Embodiment are synthesized by Invitrogen Company. See Table 4.

TABLE 5
Primers required for Embodiment 2

| Primer | Sequence (5′→3′) |
|---|---|
| 79 | CAAACAGGTGGATCGTTTTTT |
| 80 | TTATTTTTTTGTATAGCGCACCCA |
| 81 | ATGAAAAAGACAGCTATCGCG |
| 82 | AAAAAACGATCCACCTGTTTGAGCTTGGGCTACGGT |
| 83 | TACCGTAGCCCAAGCTCAAACAGGTGGATCGTTTTTT |
| 84 | CCGGAATTTTATTTTTTTGTATAGCGCACCCA |
| 85 | ATGAAATACCTATTGCCTACG |
| 86 | AAAAAACGATCCACCTGTTTGAGCCATGGCTGGTTGGGCAGC |
| 87 | CCAACCAGCCATGGCTCAAACAGGTGGATCGTTTTTT |
| 88 | CCGGAATTTTATTTTTTTGTATAGCGCACCCA |
| 89 | CGCCATATGGAACGAGCATGTGTTGCAATGAAAAAGACAGCTA |
| | TCGCG |
| 90 | CGCCATATGGAACGAGCATGTGTTGCAATGAAATACCTATTGC |
| | CTACG |
| 91 | CGCCATATGGTGGAGAGACTATGTGTGGCAGTGGTTGAAAGGG |
| | CGTGTGCGCTAGCGATGAAAAAGACAGCTATCGCG |
| 92 | CGCCATATGGTGGAGAGACTATGTGTGGCAGTGGTTGAAAGGG |
| | CGTGTGCGCTAGCGATGAAATACCTATTGCCTACG |
| 93 | CGCCATATGGTTGAAAGGTGTCTCGCGACCCTCGTGGAGAGAC |
| | TATGTGTGGCAGTGGTTGAAAGGGCGTGTGCGCTAGCG ATGA |
| | AAAAGACAGCTATCGCG |
| 94 | CGCCATATGGTTGAAAGGTGTCTCGCGACCCTCGTGGAGAGAC |
| | TATGTGTGGCAGTGGTTGAAAGGGCGTGTGCGCTAGCG ATGA |
| | AATACCTATTGCCTACG |
| 95 | CGCCATATGATGAAAAAGACAGCTATCGCG |
| 96 | CGCCATATGATGAAATACCTATTGCCTACG |

Note:
the primer No. in the table corresponds to P or SEQ ID NO.

(2) Secretory expression of dextranase BGL in LB culture medium

The secretory expression vector of the constructed dextranase BGL is transformed into the expression host BL21 (DE3) so as to obtain the recombinant strains which are named as BL21/pET-EompA-BGL and BL21/pET-EpelB-BGL. Transformants are screened from LB plate containing 100 g/mL ampicillin and plasmids are extracted for validation. See Molecular Cloning Manual for the method.

The recombinant strain is inoculated to LB liquid culture medium containing 100 μg/mL ampicillin at 37° C., and inoculated to the fresh LB culture medium (containing 100 μg/mL ampicillin) at 37° C. as per 2% inoculation amount after being cultured overnight at 200 rpm. Inducer IPTG (final concentration is 1 mmol/L) is added when OD₆₀₀ is 0.6-0.9 after being cultured at 200 rpm and collected after being induced for 6 h.

(3) Testing method of enzyme activity of dextranase

Supernatant is taken from centrifuging cells and acellular fragmentized liquid is taken from disrupted cells after the recombinant strains are fermented in a shake flask. BL21/pET-EompA-BGL and BL21/pET-EpelB-BGL fermenting supernatants and intracellular enzyme activities are tested, and the recombinant strain without enhancing small peptide motifs is taken as a negative control. See FIG. 5 (some results are selected, and sampling and testing on enzyme activity are performed after fermentation 6 h) for the enhancing effect of small peptide motifs.

Testing on enzyme activity by DNS method: take 1.0 mL of 1% (WN) barley β-glucan (dissolved in pH 6.5, 20 mmol/L Na₂HPO₄-Citrate buffer) as substrate, add 0.5 mL properly diluted enzyme liquid, react at 45° C. for 10 min, test the reducing sugar by the DNS method and take the inactivated enzyme as blank control. Definition of enzyme activity unit: the quantity of enzyme required for 1 μmol reducing sugar produced by the substrate every minute is defined as an active unit (U).

TABLE 4
Amino acid sequences and enzyme activities of
secretion enhancing small peptide motifs
involved in corresponding dextranase

| | | | Signal | Enzyme |
|---|---|---|---|---|
| n | Small peptide motif | Primer | peptide | activity |
| 1 | MERACVA | 89 | ompA | 115 |
| | | 90 | pelB | 98 |
| 2 | MERLCVAV + VERACALA | 91 | ompA | 110 |
| | | 92 | pelB | 95 |
| 3 | MERCLATL + VERLCVAV + | 93 | ompA | 134 |
| | VERACALA | 94 | pelB | 92 |
| 1 | Blank | 95 | ompA | 47 |
| | | 96 | pelB | 58 |

Note:
the primer No. in the table corresponds to P or SEQ ID NO.

(4) See Molecular Cloning Manual for the operating method for testing expression products of the recombinant strains BL21/pET-EompA-BGL and BL21/pET-EpelB-BGL. See FIG. 6 and FIG. 7 for results.

According to the common general knowledge of a person skilled in the art, one or more amino acid residue(s) are substituted for insertion or deletion of one or more amino acid residue(s) and/or the amino acids with similar properties in the strong secretory signal peptide enhancing small peptide motifs by the variant of the said strong secretory signal peptide enhancing small peptide motifs of the present invention. It belongs to a transformation or rational extension based on the present invention as well as the scope of protection of the present invention.

Similarly, polynucleotide of polypeptide, analogue or derivative having the said strong secretory signal peptide enhancing small peptide motifs of the present invention is encoded; a recombinant vector containing exogenous polynucleotide is composed of the said polynucleotide and plasmid vector of the present invention; a genetic host cell containing exogenous polynucleotide is transformed or transferred out of the said recombinant vector of present invention. All above transformations shall belong to the scope the present invention protects.

What is claimed is:

1. A strong secretory signal peptide enhancing small peptide motifs, characterized by comprising the amino acid sequence having the following formula: M(αXβYγ/αYβXγ)ₙ,
Wherein X represents an acidic amino acid;
Y represents an alkaline amino acid;
α is 0 to 2 neutral amino acid(s);
β represents 0 to 2 neutral amino acid(s);
γ represents 1 to 10 neutral amino acid(s);
n is 1 to 3.

2. The strong secretory signal peptide enhancing small peptide motifs according to claim 1, characterized in that the said acidic amino acid represented by X is Glu or Asp.

3. The strong secretory signal peptide enhancing small peptide motifs according to claim 1, characterized in that the said basic amino acid represented by Y is Arg or Lys.

4. The strong secretory signal peptide enhancing small peptide motifs according to claim 1, characterized in that the said neutral amino acid is Ala, Cys, Leu, Val, Ile or Phe.

5. The strong secretory signal peptide enhancing small peptide motifs according to claim 1, characterized in that the said n is 1.

6. The strong secretory signal peptide enhancing small peptide motifs according to claim 1, characterized in that the said α is 1 neutral amino acid, β is 0 neutral amino acid, γ is 2-5 neutral amino acids, X is Glu and Y is Arg.

7. A variant of the strong secretory signal peptide enhancing small peptide motifs according to claim 1, characterized by comprising one or more amino acid residue(s) substituted for insertion or deletion of one or more amino acid residue(s) and/or the amino acids with similar properties in the strong secretory signal peptide enhancing small peptide motifs of claim 1.

8. Polynucleotide of polypeptide, analogue or derivative having the strong secretory signal peptide enhancing small peptide motifs of claim 1 is encoded.

9. A recombinant vector containing exogenous polynucleotide, characterized in that the recombinant vector is composed of the polynucleotide and plasmid vector of claim 8.

10. A genetic host cell containing exogenous polynucleotide, characterized in that the genetic host cell is transformed or transferred out of the recombinant vector of claim 9.

11. The use of the strong secretory signal peptide enhancing small peptide motifs according to claim 1, characterized in that it is a method by using the strong secretory signal peptide enhancing small peptide motifs for constructing a vector enhancing the secretion ability of common signal peptide to improve the method for the secretory expression of exogenous proteins.
